(19) United States
(12) Patent Application Publication
(10) Pub. No.: US 2022/0315959 A1
(43) Pub. Date: Oct. 6, 2022

(54) Production of Fatty Acid Esters Using a Yeast Culture

(71) Applicant: Locus IP Company, LLC, Solon, OH (US)

(72) Inventors: Sean FARMER, Ft. Lauderdale, FL (US); Ken ALIBEK, Solon, OH (US); Trey ARNEY, Solon, OH (US); Sergey KRAVTSOV, Solon, OH (US)

(21) Appl. No.: 17/634,162

(22) PCT Filed: Aug. 10, 2020
(86) PCT No.: PCT/US2020/045605

§ 371 Date: Feb. 9, 2022

Related U.S. Application Data
(60) Provisional application No. 62/884,732, filed on Aug. 9, 2019.

(51) Int. Cl. C12P 7/6436; C12N 1/16

(52) U.S. Cl. CPC C12P 7/6436; C12N 1/16

(57) ABSTRACT

The subject invention provides improved methods for producing fatty acid esters using yeast not previously known to produce fatty acid esters. In particular, Meyerozyma spp. can be cultivated under specially-tailored conditions such that the yeast produces a variety of fatty acid esters. A yeast culture and fermentation compositions are also provided, comprising yeast cells, liquid growth medium, and a high concentration of growth by-products, such as fatty acid esters.

### CROSS-REFERENCE TO RELATED APPLICATION

This application claims priority to U.S. Provisional Patent Application No. 62/884,732, filed Aug. 9, 2019, which is incorporated herein by reference in its entirety.

### BACKGROUND OF THE INVENTION

Biologically-derived, environmentally-friendly products are becoming increasingly common in applications for industries such as, for example, oil and gas recovery; agriculture; remediation of soils, water, and other natural resources; mining; animal feed; waste treatment and disposal; food and beverage processing; and human health. For example, interest in biological amphiphilic molecules, such as microbial biosurfactants and fatty acid esters (FAEs), has been increasing in recent years due to their potential for environmentally-friendly applications.

FAEs, in particular, are molecules comprising a fatty acid combined with an alcohol. Most FAEs are produced when tallow, palm kernel oil, soya oil and/or sunflower oil are contacted with an alcohol under alkaline conditions.

FAEs are generally biodegradable and most are nontoxic chemicals with very low skin and eye irritation. They are usually considered safe for humans and the environment. Their solvency, anti-foaming properties, emulsification capabilities, and resistance to oxidation make them useful in a wide range of industries, including food processing, cosmetics, personal care products, cleaning products, paper production, water treatment, metal working, and oil production. Additionally, because FAEs have low volatility compared with many traditional solvents, they are suitable as replacements for solvents in coatings, inks and pressroom cleaners, as well as in lubricants and metalworking fluids.

Another important industry that relies on FAEs is the biodiesel industry. Biodiesel consists of a mixture of mono-alkyl esters, primarily fatty acid methyl esters (FAME), obtained from transesterification of triglycerides in plant oils or the esterification of free fatty acids using short chain alcohols. Biodiesel is primarily comprised of fatty acid methyl esters (FAME), and the demand for biodiesel as an environmentally-friendly alternative to fossil fuels is steadily increasing.

FAEs have the potential to play highly beneficial roles in, oil and gas production, agriculture, large-scale transportation and other industries that, for example, operate heavy machinery and equipment; however, efficient and sustainable methods are needed for producing the large quantities of FAEs that are required for such applications.

### BRIEF SUMMARY OF THE INVENTION

The subject invention provides materials and methods for the efficient production and use of beneficial microbes, as well as for the production and use of substances, such as metabolites, derived from these microbes and the substrate in which they are produced.

In particular, the subject invention provides materials and methods for producing fatty acids esters using a microorganism that was previously not understood to be useful for producing multiple types of fatty acids esters. Advantageously, the subject invention increases efficiency and reduces costs associated with FAE production, compared to traditional production methods.

In general, the subject methods involve cultivating a yeast strain under specially-tailored conditions that influence one or more biological mechanisms, which, when activated in the yeast, result in the unnatural production of FAEs.

In specific embodiments, the methods utilize a yeast, preferably, a Meyerozyma sp., more preferably, Meyerozyma guilliermondii. Meyerozyma guilliermondii was previously not known to possess the capability for producing a variety of FAEs; thus, the methods of the subject invention provide for the unexpected and advantageous result of non-natural, FAE production.

In one embodiment, the method comprises inoculating a cultivation reactor comprising a liquid growth medium with a yeast to produce a yeast seed culture; and cultivating the yeast see culture under conditions that are favorable for logarithmic growth of the yeast to produce a yeast inoculum.

The yeast seed culture can be grown for an amount of time and at a temperature that allows for the development of a concentration of yeast cells necessary to seed a large scale fermentation reactor for the production of FAEs using fermentation. In certain embodiments, cultivation time is about 30 hours to about 54 hours at a temperature of about 20° C. to about 30° C.

In one embodiment, the method comprises inoculating a fermentation reactor comprising a liquid growth medium with the yeast inoculum to produce a yeast culture and cultivating the yeast culture under conditions that are favorable for fermentation to produce FAEs.

The yeast culture can be grown for an amount of time that allows for the production of a desired concentration of FAEs. In certain embodiments, the fermentation time is about 48 hours to about 102 hours, preferably 72 hours to 102 hours.

In one embodiment, the conditions favorable for growing yeast and the ensuing production of FAEs include specific ranges of temperature, dO₂ concentration, and pH.

In one embodiment, a favorable temperature is about 20° C. to about 30° C., or about 22° C. to about 24° C. In one embodiment, a favorable dO₂ concentration for fermentation is about 30% to about 65%. In one embodiment, a favorable pH is about 4.0 to about 8.0, or about 5.0 to about 7.5.

In certain embodiments, the liquid growth medium can also be specially-tailored to permit rapid yeast growth and promote and/or induce the production of FAEs. The growth medium can comprise a specific combination of sources of, for example, proteins, amino acids, antioxidants, vitamins, minerals, nitrogen, potassium, phosphorous, magnesium, calcium, sodium, carbon, salts, pH adjusters, and/or other trace elements.

In some embodiments, the growth medium and pH adjuster can be replenished throughout cultivation.

In certain embodiments, the methods of the subject invention utilize solid state fermentation (SSF), submerged fermentation, or modified and/or combined versions thereof In preferred embodiments, the method utilizes submerged fermentation.

The methods can be scaled up or down. Most notably, the methods can be scaled to an industrial scale, i.e., a scale that is suitable for use in supplying FAEs in amounts to meet the demand for commercial applications, e.g,. biodiesel production.

The subject methods can be useful for producing microbial growth by-products, such as FAEs. These FAEs can be collected throughout the fermentation process or at specific time points. In some embodiments, fatty acids can be collected along with FAEs.

In specific embodiments, the FAEs include fatty acid methyl ester, fatty acid ethyl ester, fatty acid propyl ester, fatty acid butyl ester, fatty acid hexyl ester, and/or fatty acid isopropyl ester.

The microbial growth by-products produced according to the subject invention can be retained in the cells of the microorganisms and/or secreted into the liquid medium in which the microbes are growing.

In certain embodiments of the subject methods, an alcohol is added after fermentation to increase the concentration of FAEs in the composition. In certain embodiments, the alcohol is mixed with a composition resulting from fermentation for about 15 minutes to about 30 hours, 45 minutes to about 4 hours, or about 1 hour.

Resulting layers can be recovered from the culture simply by mechanical collection, and, if desired, can be purified according to known methods.

The yeast culture, including the biomass and residual fermentation medium, can also be processed for further extraction in order to recover, for example, FAEs and/or other microbial growth by-products produced during cultivation. Processing can comprise, for example, centrifugation and/or washing, or other known methods, and can include optional further purification.

In certain embodiments, the subject invention provides microbe-based products. The microbe-based products can comprise the entire culture produced according to the subject methods, including the microorganisms and/or their growth by-products as well as residual growth medium and/or nutrients. The microorganisms can be alive, viable, or in an inactive form. They can be in the form of a biofilm, vegetative cells, and/or a combination thereof In certain embodiments, no microbes are present, wherein the composition comprises microbial growth by-products, e.g., FAEs, which have been extracted from the culture and, optionally, purified.

### DETAILED DESCRIPTION

The subject invention provides materials and methods for the efficient production and use of beneficial microbes, as well as for the production and use of substances, such as metabolites, derived from these microbes and the substrate in which they are produced.

In particular, the subject invention provides materials and methods for producing fatty acid esters (FAEs) using a microorganism that was previously not known to be useful for producing multiple types of FAEs. Advantageously, the subject invention increases efficiency and reduces costs associated with FAE production, compared to traditional production methods.

In general, the subject methods involve cultivating a yeast strain under specially-tailored conditions that influence one or more biological mechanisms, which, when activated in the yeast, result in the unnatural production of FAEs. In specific embodiments, the methods utilize a yeast, preferably Meyerozyma guilliermondii.

### Selected Definitions

As used herein, a “biofilm” is a complex aggregate of microorganisms, wherein the cells adhere to each other and produce extracellular substances that encase the cells. Biofilms can also adhere to surfaces. The cells in biofilms are physiologically distinct from planktonic cells of the same organism, which are single cells that can float or swim in liquid medium.

As used herein, an “isolated” or “purified” nucleic acid molecule, polynucleotide, polypeptide, protein or organic compound such as a small molecule (e.g., those described below), is substantially free of other compounds, such as cellular material, with which it is associated in nature. An isolated microbial strain means that the strain is removed from the environment in which it exists in nature. Thus, the isolated strain may exist as, for example, a biologically pure culture, in association with a carrier.

In certain embodiments, purified compounds are at least 60% by weight the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, high-performance liquid chromatography (HPLC) analysis, or gas chromatography.

As used herein, an “isomer” refers to a molecule with an identical chemical formula to another molecule, but having unique structures. Isomers can be constitutional isomers, in which atoms and functional groups are bonded at different locations and stereoisomers (spatial isomers), in which the bond structure is the same, but the geometrical positioning of atoms and functional groups in space is different. FAE isomers, for example, can differ in the cis or trans confirmation of double bonded carbon atoms in the fatty acid chain.

An “analog” of a molecule does not have an identical chemical formula, but can have similar structure and/or functions. A “structural analog” or “chemical analog” is a compound having a structure that is similar to that of another compound, but having one or more differing components, such as one or more different atoms, functional groups, or substructures. As used herein, “functional analogs,” are compounds that have similar physical, chemical, biochemical, or pharmacological properties. Despite their similarities, however, chemical analogs can be, but are not always, functional analogs, and functional analogs can be, but are not always, chemical analogs.

A “metabolite” refers to any substance produced by metabolism (e.g., a growth by-product) or a substance necessary for taking part in a particular metabolic process. A metabolite can be an organic compound that is a starting material, an intermediate in, or an end product of metabolism. Examples of a metabolite include, but are not limited to, biosurfactants, enzymes, biopolymers, fatty acids, bioemulsifiers, acids, solvents, amino acids, nucleic acids, peptides, proteins, lipids, carbohydrates, vitamins, and/or minerals.

The systems and methods of the subject invention can be used to produce microbe-based compositions. As used herein, a “microbe-based composition” is a composition that comprises components that were produced as the result of the growth of microorganisms or other cell cultures. Thus, the microbe-based composition may comprise the microbes themselves and/or by-products of microbial growth. The microbes may be in a vegetative state, in a spore foi in, or a mixture of these.

The microbes may be planktonic or in a biofilm form, or a mixture of both. The by-products of growth may be, for example, metabolites, cell membrane components, synthesized proteins, and/or other cellular components. The microbes may be intact or lysed. In some embodiments, the microbes are removed from the microbe-based composition. In some embodiments the microbes are present, with the medium in which they were grown, in the microbe-based composition. The cells may be present at, for example, a concentration of at least 1×10⁴, 1×10⁵, 1×10⁶, 1×10⁷, 1×10⁸, 1×10⁹, 1×10¹⁰, 1×10¹¹, 1×10¹², or 1×10¹³ or more cells per gram or milliliter of the composition.

The subject invention further provides “microbe-based products,” which are products that are to be applied in practice to achieve a desired result. The microbe-based product can be simply the microbe-based composition harvested from the microbe cultivation process. Alternatively, the microbe-based product may comprise only a portion of the product of cultivation (e.g., only the growth by-products), and/or the microbe-based product may comprise further ingredients that have been added. These additional ingredients can include, for example, stabilizers; buffers; appropriate carriers, such as water or salt solutions; added nutrients to support further microbial growth; non-nutrient growth enhancers, such as amino acids, and/or agents that facilitate tracking of the microbes; and/or the composition in the environment to which it is applied. The microbe-based product may also comprise mixtures of microbe-based compositions. The microbe-based product may also comprise one or more components of a microbe-based composition that have been processed in some way such as, but not limited to, filtering, centrifugation, lysing, drying, purification and the like.

As used herein “reduction” means a negative alteration, and “increase” means a positive alteration, wherein the negative or positive alteration is at least 1%, 5%, 10%, 25%, 50%, 75%, or 100%.

As used herein, “surfactant” means a surface-active compound that lowers the surface tension (or interfacial tension) between two liquids, between a liquid and a gas, or between a liquid and a solid. Surfactants act as, e.g., detergents, wetting agents, emulsifiers, foaming agents, and dispersants. A “biosurfactant” is a surface-active substance produced by a living cell.

Ranges provided herein are understood to be shorthand for all of the values within the range. For example, a range of 1 to 20 is understood to include any number, combination of numbers, or sub-range from the group consisting of 1, 2, 3, 4, 5, 6, 7, 8, 9, 10, 11, 12, 13, 14, 15, 16, 17, 18, 19, or 20 as well as all intervening decimal values between the aforementioned integers such as 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

The transitional term “comprising,” which is synonymous with “including” or “containing,” is inclusive or open-ended and does not exclude additional, unrecited elements, or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention. Use of the term “comprising” contemplates other embodiments that “consist” or “consist essentially” of the recited component(s).

Unless specifically stated or obvious from context, as used herein, the term “or” is understood to be inclusive. Unless specifically stated or obvious from context, as used herein, the terms “a,” “an,” and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

Other features and advantages of the invention will be apparent from the following description of the preferred embodiments thereof and from the claims. All references cited herein are hereby incorporated by reference.

### Methods for Producing Fatty Acid Esters

The subject invention provides methods for the cultivation of microorganisms and the production of microbial metabolites and/or other by-products of microbial growth using solid state fermentation, submerged fermentation, or a combination thereof. In preferred embodiments, cultivation is carried out using submerged fermentation.

As used herein, “fermentation” refers to growth of cells under controlled conditions. The growth could be aerobic or anaerobic.

In one embodiment, the subject invention provides materials and methods for the production of biomass (e.g., viable cellular material), extracellular metabolites, residual nutrients, and/or intracellular components.

The methods can be scaled up or down in size. Most notably, the methods can be scaled to an industrial scale, i.e., a scale that is suitable for use in supplying FAEs in amounts to meet the demand for commercial applications, for example, the production of compositions for enhanced oil recovery.

In specific embodiments, the subject invention provides materials and methods for producing multiple FAEs using one type of microorganism. Advantageously, the subject invention increases efficiency and reduces the costs associated with FAE production, compared to traditional production methods.

The general structure of a FAE comprises an alcohol group covalently linked to one or more fatty acids. The fatty acid chain(s) of the FAEs can comprise 6 to 22 carbons, 8 to 20 carbons, 10 to 18 carbons, or 12 to 16 carbons. The fatty acids can be saturated or unsaturated, with one or more double bonds in the carbon chain or in various isomers.

The FAEs can include, for example, fatty acid methyl ester (FAME), fatty acid ethyl ester, fatty acid propyl ester, fatty acid butyl ester, fatty acid isopropyl ester, and/or fatty acid hexyl ester.

The subject methods involve cultivating a yeast strain under specially-tailored conditions that influence one or more biological mechanisms, which, when activated in the yeast, result in the unnatural production of FAEs. In certain embodiments, the one or more biological mechanisms are inactive or weakly active in the yeast, absent these influencing conditions.

The microorganisms produced according to the subject invention can be natural or genetically modified microorganisms. For example, the microorganisms may be transformed with specific genes to exhibit specific characteristics. The microorganisms may also be mutants of a desired strain. As used herein, “mutant” means a strain, genetic variant, or subtype of a reference microorganism, wherein the mutant has one or more genetic variations (e.g., a point mutation, missense mutation, nonsense mutation, deletion, duplication, frameshift mutation, or repeat expansion) as compared to the reference microorganism. Procedures for making mutants are well known in the microbiological art. For example, UV mutagenesis and methylnitronitrosoguanidine are used extensively toward this end.

In specific embodiments, the methods utilize Meyerozyma guilliermondii. Meyerozyma guilliermondii was previously not known to be useful for producing multiple FAEs; thus, the methods of the subject invention provide for the unexpected and advantageous result of non-natural FAE production.

In one embodiment, the yeast that will be used for the production of FAEs undergoes an initial cultivation to provide a starter culture for a subsequent fermentation process. The method comprises inoculating a cultivation or fermentation reactor comprising a liquid growth medium with a yeast, preferably Meyerozyma guilliermondii, to produce a yeast seed culture; and cultivating the yeast seed culture under conditions favorable for logarithmic growth to achieve a desired concentration of cells that will serve as a yeast inoculum for the ensuing fermentation procedure.

In one embodiment, the yeast seed culture can be cultivated for an amount of time and temperature that allows for the development of a concentration of yeast cells to be used to seed a large scale culture for the production of FAE using fermentation. In certain embodiments, the cultivation time is about 30 hours to about 54 hours at a temperature of about 20° C. to about 30° C. The pH, dO₂ concentration, and growth media required to effectively grow yeast to a high density are well known in the art.

In certain embodiments, the subject method comprises inoculating a fermentation reactor comprising a liquid growth medium with the yeast inoculum produced according to the above description to produce a yeast culture; and cultivating the yeast culture under conditions favorable for production of FAEs.

In some embodiments, the yeast inoculum can be pre-mixed with water and/or a liquid growth medium, if desired.

The microbe growth vessel used according to the subject invention can be any fermentation reactor for industrial use. In one embodiment, the vessel may have functional controls/sensors or may be connected to functional controls/sensors to measure important factors in the cultivation process, such as, for example, pH, oxygen, pressure, temperature, agitator shaft power, humidity, viscosity, microbial density, and/or metabolite concentration.

In a further embodiment, the vessel may also be able to monitor the growth of microorganisms inside the vessel (e.g., measurement of cell number and growth phases). Alternatively, samples may be taken from the vessel and subjected to enumeration and/or purity measurement techniques known in the art, such as dilution plating technique. For example, in one embodiment, sampling of the cells can occur at the initiation of fermentation and then at least once daily thereafter, and/or at the time of harvesting microbes and/or the practitioner can sample microbial growth by-products from the reactor.

In certain embodiments, the cultivation method utilizes submerged fermentation in a liquid growth medium. In one embodiment, the liquid growth medium comprises a carbon source. The carbon source can be a carbohydrate, such as glucose, sucrose, lactose, fructose, trehalose, mannose, mannitol, and/or maltose; organic acids such as acetic acid, fumaric acid, citric acid, propionic acid, malic acid, malonic acid, and/or pyruvic acid; alcohols such as ethanol, propanol, butanol, pentanol, hexanol, isobutanol, and/or glycerol; fats and oils such as soybean oil, rice bran oil, olive oil, corn oil, sesame oil, and/or linseed oil; powdered molasses, etc. These carbon sources may be used independently or in a combination of two or more.

In one embodiment, the liquid growth medium comprises a nitrogen source. The nitrogen source can be, for example, potassium nitrate, ammonium nitrate, ammonium sulfate, ammonium phosphate, ammonia, urea, and/or ammonium chloride. These nitrogen sources may be used independently or in a combination of two or more.

In one embodiment, one or more inorganic salts may also be included in the liquid growth medium. Inorganic salts can include, for example, potassium phosphate monobasic, potassium phosphate dibasic, disodium hydrogen phosphate, potassium chloride, magnesium sulfate, magnesium chloride, iron sulfate, iron chloride, manganese sulfate, manganese chloride, zinc sulfate, lead chloride, copper sulfate, calcium chloride, calcium carbonate, calcium nitrate, magnesium sulfate, sodium phosphate, sodium chloride, and/or sodium carbonate. These inorganic salts may be used independently or in a combination of two or more.

In one embodiment, growth factors and trace nutrients for microorganisms are included in the medium. This is particularly preferred when growing microbes that are incapable of producing all of the vitamins they require. Inorganic nutrients, including trace elements such as iron, zinc, copper, manganese, molybdenum, and/or cobalt may also be included in the medium. Furthermore, sources of vitamins, essential amino acids, proteins, and microelements can be included, for example, corn flour, peptone from casein, gelatin, meat, pea, porcine, potatoes, soybean, wheat, or other sources, yeast extract, potato extract, beef extract, soybean extract, banana peel extract, and the like, or in purified forms. Amino acids such as, for example, those useful for biosynthesis of proteins, can also be included.

The method of cultivation can further provide oxygenation to the growing culture. One embodiment utilizes slow motion of air to remove low-oxygen containing air and introduce oxygenated air. The oxygenated air may be ambient air supplemented daily through mechanisms including impellers for mechanical agitation of the liquid and air spargers for supplying bubbles of air into the liquid for the dissolution of oxygen. In certain embodiments, dissolved oxygen (DO) levels are maintained at about 25% to about 75%, about 30% to about 70%, about 35% to about 65%, about 40% to about 60%, or about 50% of air saturation. Air flow can be supplied at, for example, about 0.5 to about 2.0 v/m or about 1.0 to about 1.5 v/m.

In some embodiments, the method for cultivation may further comprise adding additional acids and/or antimicrobials in the liquid medium before and/or during the cultivation process. Antimicrobial agents or antibiotics are used for protecting the culture against contamination. In some embodiments, however, the metabolites produced by the yeast culture provide sufficient antimicrobial effects to prevent contamination of the culture. Antimicrobial agents can include, for example, streptomycin, spectinomycin, tetracycline, oxytetracycline, doxycycline, kanamycin, ampicillin, azithromycin, clarithromycin, erythromycin, gentamicin, neomycin, and vancomycin.

In one embodiment, prior to inoculation, the components of the liquid culture medium can be sterilized. If used, an anchoring carrier is also preferably sterilized, for example, using an autoclave or other method known in the art. Additionally, water used for preparing the medium can be filtered to prevent contamination.

In one embodiment, sterilization of the liquid growth medium can be achieved by placing the components of the liquid culture medium in water at a temperature of about 85° C. to about 100° C. In one embodiment, sterilization can be achieved by dissolving the components in about 1% to about 3% hydrogen peroxide in a ratio of 1:3 (w/v).

In one embodiment, the equipment used for cultivation is sterile. The cultivation equipment, such as the reactor/vessel, may be separated from, but connected to, a sterilizing unit, e.g., an autoclave. The cultivation equipment may also have a sterilizing unit that sterilizes in situ before starting the inoculation. Air can be sterilized by methods know in the art. For example, the ambient air can pass through at least one filter before being introduced into the vessel. In other embodiments, the medium may be pasteurized or, optionally, no heat at all added, in which the use of pH and/or low water activity may be exploited to control unwanted microbial growth.

The pH of the mixture should be suitable for the microorganism that is being produced. In some embodiments, the pH is about 2.0 to about 11.0, about 3.0 to about 10.0, about 4.0 to about 9.0, about 5.0 to about 8.0, or about 5.0 to about 7.5. In one embodiment, pH regulators, such as carbonates and phosphates, may be used to stabilize pH near a preferred value. In certain embodiments, a base solution is used to adjust the pH of the yeast culture to a favorable level, for example, a 15% to 30% or a 20% to 25% NaOH solution. The base solution can be included in the growth medium, and/or it can be fed into the fermentation reactor during cultivation to adjust the pH, for example, after about 24 hours. Alternatively, the pH can be adjusted by the practitioner using HCl.

In one embodiment, the method of cultivation is carried out at about 5° C. to about 100° C., about 15° C. to about 60° C., about 18° C. to about 45° C., or about 20° C. to about 30° C. In one embodiment, the cultivation may be carried out continuously at a constant temperature. In another embodiment, the cultivation may be subject to changing temperatures.

According to the subject methods, the yeast can be incubated in the fermentation system for a time period sufficient to achieve a desired effect, e.g., production of a desired amount of cell biomass or a desired amount of one or more microbial growth by-products. The biomass content may be, for example from .01 g/1 to 1000 g/1 or more, or from 0.1 g/1 to 500 g/l.

In certain embodiments, fermentation occurs for about 12 hours to about 144 hours, for about 48 hours to about 132 hours, for about 72 hours to about 120 hours, for about 90 hours to about 102 hours, or for about 94 to about 98 hours.

The microbial growth by-product produced by microorganisms of interest may be retained in the microorganisms or secreted into the growth medium. In some embodiments, a layer is produced during cultivation that can comprise some microbial growth by-products, such as fatty acids and FAEs, which may be collected continuously throughout the fermentation process, at specific time points, and/or at the completion of the fermentation process. This layer can be in a liquid form, foam form, or a combination of both forms.

At the end of the fermentation cycle, in some embodiments, the yeast culture is left to settle for about 20 to 28 hours. The yeast culture can be kept in the fermentation reactor, or it can be transferred to a separate container for settling.

As the yeast culture settles, an aqueous layer, which can include cell biomass, settles to the bottom of the reactor, and a FAE layer forms and on top of the aqueous layer.

In some embodiments, the FAE layer comprises a concentration of fatty acids and FAEs of about 1% to about 25%, about 5% to about 20%, or 10% to about 14%. The FAE layer can be harvested and, optionally, concentrated, and/or purified.

In some embodiments, the collected FAE layer or the composition that results from the fermentation process is mixed with alcohol, such as methanol, ethanol, propanol or isopropyl alcohol. This composition is then mixed for about 15 minutes to about 30 hours, 45 minutes to about 4 hours, or about 1 hour. After the mixing, the composition settles, and a layer of FAEs forms above an aqueous layer. The aqueous layer can be removed. The layers can be recovered from the culture simply by mechanical collection, and, if desired, can be purified according to known methods.

The FAE layer can be processed by, e.g., washing and/or centrifuging, to extract the microbial growth by-products. Optionally, the growth by-products can then be stored, purified, and/or used directly in crude form. In one embodiment, the growth medium may contain compounds that stabilize the activity of the microbial growth by-product.

In some embodiments, the FAE layer is mixed with 10% hexanol, resulting in formation of a hexanol phase comprising isopropyl palmitic acid esters. By evaporating the hexanol phase, isopropyl palmitic acid esters with a purity of at least 95%, preferably at least 98%, can be achieved.

The yeast culture, including the biomass and residual fermentation medium, as well as any foam produced, can also be processed for further extraction in order to recover, for example, FAEs and/or other microbial growth by-products produced during cultivation. Processing can comprise, for example, centrifugation and washing, or other known methods, and can include purification.

The subject methods can be used to produce high concentrations of FAEs, for example, about 0.5 g/L to about 100 g/L, about 1 g/L to about 75 g/L, about 2 g/L to about 50 g/L, about 3 g/L to about 25 g/L, about 4 g/L to about 20 g/L, or about 5 g/L to about 15 g/L of fatty acid methyl ester, fatty acid ethyl ester, fatty acid propyl ester, fatty acid butyl ester, fatty acid hexyl ester and/or fatty acid isopropyl ester.

The methods can be performed in a batch, quasi-continuous, or continuous processes. In one embodiment the entire yeast culture is removed upon the completion of cultivation (e.g., upon achieving a desired cell density or metabolite concentration). In this batch procedure, an entirely new batch is initiated upon harvesting of the first batch.

In another embodiment, only a portion of the culture is removed at any one time. In this manner, a continuous or quasi-continuous system is created. The composition that is removed can be a cell-free liquid, and/or it can contain some cells.

### Preparation of Microbe-Based Products

In certain embodiments, the subject invention provides microbe-based products, as well as their uses in a variety of settings including, for example, oil and gas recovery; bioremediation and mining; waste disposal and treatment; animal health (e.g., livestock production and aquaculture); plant health and productivity (e.g., agriculture, horticulture, crops, pest control, forestry, turf management, and pastures); and human health (e.g., probiotics, pharmaceuticals and cosmetics).

One microbe-based product of the subject invention is simply a yeast culture comprising yeast cells, a growth medium, and one or more FAEs. The one or more FAEs can be retained in the cells of the yeast and/or present as a secretion in the growth medium, e.g., in the form of a FAE layer or a foam layer. The yeast culture can also comprise other metabolites produced by the yeast.

The microbe-based products may be used without further stabilization, preservation, and storage. Advantageously, direct usage of the microbe-based products preserves a high viability of the microorganisms, reduces the possibility of contamination from foreign agents and undesirable microorganisms, and maintains the activity of the by-products of microbial growth.

If present in the microbe-based product, the microorganisms may be in an active or inactive form. The amount of biomass in the composition, by weight, may be, for example, from 0% to 100%, 10% to 90%, 20% to 80%, 30% to 70%, or 40% to 60%, inclusive of all percentages therebetween.

In one embodiment, the composition does not comprise living microorganisms. In one embodiment, the composition does not comprise microorganisms, whether living or inactive.

The product of fermentation may be used directly without extraction or purification. If desired, however, extraction and purification can be easily achieved. In some embodiments, all or a portion of the entire culture, including the FAEs, can be harvested from the vessel and then processed to recover the FAEs. For example, in some embodiments, the culture is centrifuged to remove the yeast cells and then subjected to known extraction and, optionally, purification methods to recover the FAEs. All or a portion of the product can also be dried and later dissolved in water.

In some embodiments, extraction does not require solvents. In some embodiments, standard extraction methods or techniques known to those skilled in the art, including those that use solvents, can be employed.

Advantageously, in accordance with the subject invention, the microbe-based product may comprise the substrate in which the microbes were grown. In one embodiment, the composition may be, for example, at least 0.1%, 0.05%, 1%, 5%, 10%, 25%, 50%, 75%, or 100%, by weight, growth medium.

In one embodiment, the compositions comprise one or more microbial growth by-products, wherein the growth by-product has been extracted from the culture and, optionally, purified. For example, in one embodiment, the composition comprises a FAE layer that forms during fermentation and rises to the top of the culture. This layer can be extracted and then subjected to known purification methods.

In one exemplary embodiment, extraction and purification comprises mixing a fatty acid layer obtained from the subject fermentation methods and mixing it with 10% hexanol. The hexanol extracts fatty acid isopropyl esters, e.g., isopropyl palmitic acid ester, to a high degree of purity, e.g., at least 95%.

In a specific preferred embodiment, the composition comprises FAE molecules, such as those described above. For example, the yeast culture can comprise about 0.5 g/L to about 100 g/L, about 1 g/L to about 75 g/L, about 2 g/L to about 50 g/L, about 3 g/L to about 25 g/L, about 4 g/L to about 20 g/L, or about 5 g/L to about 15 g/L of fatty acid methyl ester, fatty acid ethyl ester, fatty acid propyl ester, fatty acid butyl ester, and/or fatty acid hexyl ester.

In certain embodiments, the use of crude/unpurified forms of microbial growth by-products according to the subject invention can have advantages over, for example, purified microbial metabolites alone, due to, for example, the use of the entire culture. Additionally, the compositions can comprise a variety of microbial metabolites (e.g., biosurfactants, enzymes, acids, and solvents) in the culture that may work in synergy with one another to achieve a desired effect.

In some embodiments, the composition can be placed in containers of appropriate size, taking into consideration, for example, the intended use, the contemplated method of application, the size of the fermentation vessel, and any mode of transportation from a microbe growth facility to the location of use. Thus, the containers into which the microbe-based composition is placed may be, for example, from 1 gallon to 2,000 gallons or more. In certain embodiments the containers are 2 gallons, 5 gallons, 25 gallons, 250 gallons, 2000 gallons, or larger.

The microbe-based product can be removed from the container and transferred to the site of application via, for example, tanker for immediate use.

Upon harvesting the microbe-based composition from the growth vessels, further components can be added as the harvested product is placed into containers and/or piped (or otherwise transported for use). The additives can be, for example, buffers, carriers, other microbe-based compositions produced at the same or different facility, viscosity modifiers, preservatives, nutrients for microbe growth, tracking agents, pesticides, and other ingredients specific for an intended use.

Optionally, the product can be stored prior to use. The storage time is preferably short. Thus, the storage time may be less than 60 days, 45 days, 30 days, 20 days, 15 days, 10 days, 7 days, 5 days, 3 days, 2 days, 1 day, or 12 hours. In a preferred embodiment, if live cells are present in the product, the product is stored at a cool temperature such as, for example, less than 20° C., 15° C., 10° C., or 5° C. On the other hand, a FAE composition can typically be stored at ambient temperatures if sealed in an airtight container.

### Methods of Use

The compositions produced according to the methods of the subject invention can be used for a variety of purposes. In one embodiment, the composition can be used in agriculture. For example, methods are provided wherein a composition produced according to the subject invention is applied to a plant and/or its environment to treat and/or prevent the spread of pests and/or diseases. The composition can also be useful for enhancing water dispersal and absorption in the soil, as well as enhance nutrient absorption from the soil through plant roots, facilitate plant health, increase yields, increase carbon sequestration, and/or manage soil aeration.

In one embodiment, the subject compositions can be utilized as biodiesel or another form of biofuel to power heavy machinery and transport vehicles.

In one embodiment, the subject compositions can be utilized in the formulation of lubricants for metalworking, engines, and other heavy metal machinery.

In one embodiment, the subject compositions can be utilized as a solvent, emulsifier, anti-foam additive, or thickener for a cosmetic or therapeutic cream, lotion or ointment.

In one embodiment, the subject compositions can be utilized as an oil well treatment or some other treatment for the oil and gas industry. When applied to an oil well, wellbore, subterranean formation, or to equipment used for recovery oil and/or gas, the compositions produced according to the subject invention can be used in methods for enhancement of crude oil recovery; reduction of oil viscosity; removal and dispersal of paraffin from rods, tubing, liners, and/or pumps; prevention of equipment corrosion; recovery of oil from oil sands and stripper wells; enhancement of fracking operations as fracturing fluids; reduction of H₂S concentration in formations and crude oil; and/or cleaning of tanks, flowlines, and pipelines.

In one embodiment, the compositions produced according to the subject invention can be used to improve one or more properties of oil. For example, methods are provided wherein the composition is applied to oil or to an oil-bearing formation in order to reduce the viscosity of the oil, convert the oil from sour to sweet oil, and/or to upgrade the oil from heavy crude into lighter fractions.

In one embodiment, the compositions produced according to the subject invention can be used to clean industrial equipment. For example, methods are provided wherein a composition is applied to oil production equipment such as an oil well rod, tubing and/or casing, to remove heavy hydrocarbons, paraffins, asphaltenes, scales and other contaminants from the equipment. The composition can also be applied to equipment used in other industries, for example, food processing and preparation, agriculture, paper milling, and others where fats, oils, and greases build up and contaminate and/or foul the equipment.

In one embodiment, the compositions produced according to the subject invention can be used to enhance animal health. For example, methods are provided wherein the composition can be applied to animal feed or water, or mixed with the feed or water, and used to prevent the spread of disease in livestock and aquaculture operations, reduce the need for antibiotic use in large quantities, as well as to provide supplemental fatty acids, proteins and other nutrients.

In one embodiment, the compositions produced according to the subject invention can be used to prevent spoilage of food, prolong the consumable life of food, and/or to prevent food-borne illnesses. For example, methods are provided wherein the composition is applied to a food product, such as fresh produce, baked goods, meats, and post-harvest grains, to prevent undesirable microbial growth.

Other uses for the subject compositions include, but are not limited to, biofertilizers, biopesticides, bioleaching, bioremediation of soil and/or water, pharmaceutical adjuvants (e.g., for increasing bioavailability of orally ingested drugs), and control of unwanted microbial growth.

### Local Production of Microbe-Based Products

In certain embodiments of the subject invention, a microbe growth facility produces fresh, high-density microorganisms and/or microbial growth by-products of interest, i.e., FAEs, on a desired scale. The microbe growth facility may be located at or near the site of application. The facility produces high-density microbe-based compositions in batch, quasi-continuous, or continuous cultivation.

The microbe growth facilities of the subject invention can be located at the location where the microbe-based product will be used. For example, the microbe growth facility may be less than 300, 250, 200, 150, 100, 75, 50, 25, 15, 10, 5, 3, or 1 mile from the location of use.

Because the microbe-based product can be generated locally, without resort to the microorganism stabilization, preservation, storage, and transportation processes of conventional microbial production, a much higher density of microorganisms can be generated, thereby requiring a smaller volume of the microbe-based product for use in the on-site application or allows for higher density microbial applications where necessary to achieve the desired efficacy. This makes the system efficient and can eliminate the need to stabilize cells or separate them from their culture medium. Local generation of the microbe-based product also facilitates the inclusion of the growth medium in the product. The medium can contain agents produced during fermentation that are particularly well-suited for local use.

Locally-produced high density, robust cultures of microbes are more effective in the field than those that have remained in the supply chain for some time. The microbe-based products of the subject invention are particularly advantageous compared to traditional products wherein cells have been separated from metabolites and nutrients present in the fermentation growth media. Reduced transportation times allow for the production and delivery of fresh batches of microbes and/or their metabolites at the time and volume as required by local demand.

The microbe growth facilities of the subject invention produce fresh, microbe-based compositions, comprising the microbes themselves, microbial metabolites, and/or other components of the medium in which the microbes are grown. If desired, the compositions can have a high density of vegetative cells or propagules, or a mixture of vegetative cells and propagules.

In one embodiment, the microbe growth facility is located on, or near, a site where the microbe-based products will be used, for example, within 300 miles, 200 miles, or even within 100 miles. Advantageously, this allows for the compositions to be tailored for use at a specified location. The formula and potency of microbe-based compositions can be customized for a specific application and in accordance with the local conditions at the time of application.

Advantageously, distributed microbe growth facilities provide a solution to the current problem of relying on far-flung industrial-sized producers whose product quality suffers due to upstream processing delays, supply chain bottlenecks, improper storage, and other contingencies that inhibit the timely delivery and application of, for example, a viable, high cell-count product and the associated medium and metabolites in which the cells are originally grown.

Furthermore, by producing a composition locally, the formulation and potency can be adjusted in real time to a specific location and the conditions present at the time of application. This provides advantages over compositions that are pre-made in a central location and have, for example, set ratios and formulations that may not be optimal for a given location.

The microbe growth facilities provide manufacturing versatility by their ability to tailor the microbe-based products to improve synergies with destination geographies. Advantageously, in preferred embodiments, the systems of the subject invention harness the power of naturally-occurring local microorganisms and their metabolic by-products.

Local production and delivery within, for example, 24 hours of fermentation results in pure, high cell density compositions and substantially lower shipping costs. Given the prospects for rapid advancement in the development of more effective and powerful microbial inoculants, consumers will benefit greatly from this ability to rapidly deliver microbe-based products.

### EXAMPLE

A greater understanding of the present invention and of its many advantages may be had from the following example, given by way of illustration. The following example is illustrative of some of the methods, applications, embodiments and variants of the present invention. They are not to be considered as limiting the invention.

### Example 1

### Production of Fatty Acid Esters

Meyerozyma guilliermondii inoculum is grown in a small-scale reactor for 36 to 48 hours. A cultivation reactor with 25 mL of sterilized, deionized water and a growth medium is inoculated with the M. guilliermondii cells to produce a yeast culture. The growth medium comprises:

| Ingredient | Concentration | |
|---|---|---|
| Dextrose | 30 | g/L |
| Yeast Extract | 5 | g/L |
| Urea | 2 | g/L |
| Calcium Carbonate | 1 | g/L |
| Sodium Chloride | 0.1 | g/L |

The temperature is maintained at about 22° C. to about 28° C. and dO₂ is about 35% to about 65%.

Sampling for CFU concentration and/or purity is performed at 0 hr, then on day 2 and day 3 during this initial cultivation. Once the culture achieves the desired, uncontaminated CFU, then it is used as an inoculum for a 2,000 liter reactor.

A fermentation reactor with 150 gallons of sterilized, deionized water and a growth medium is inoculated with the yeast inoculum from above to produce a yeast culture. The growth medium comprises:

| Ingredient | Concentration | |
|---|---|---|
| Ammonium Nitrate | 10 | g/L |
| Potassium Phosphate Dibasic | 2.5 | g/L |
| Sodium Phosphate monobasic | 0.15 | g/L |
| Magnesium Sulfate | 0.5 | g/L |
| Calcium Chloride | 0.1 | g/L |
| Manganese Sulfate | 0.02 | g/L |
| Casein Peptone | 1 | g/L |
| Dextrose | 5 | g/L |
| Soybean Oil | 10 | ml/L |

A 4L solution with 300 g of streptomycin and 20 g of oxytetracycline is added to the fermentation reactor. Before the initial 22-24 hours of fermentation, the pH is adjusted to 5.5 using an aqueous HCl solution. The temperature is initiated and then maintained at 24° C. and the dO₂ remains at about 35% to about 65% during fermentation. After the initial 22-26 hours of fermentation, an aqueous base solution comprising 20% NaOH is fed into the reactor to adjust and maintain pH automatically at about 6.95 to about 7.05. Sampling for the amount of soybean oil is performed every day during fermentation. If the FAEs are foaming out of the liquid composition, the foam is collected continuously throughout the fermentation process.

Total fermentation time is about 94 to about 98 hours. After the cycle ends, the yeast culture is placed into a tank and allowed to settle for up to 24 hours. An upper layer containing FAEs is separated from a lower, aqueous layer. Alcohols, such as methanol, ethanol, propanol, butanol, and isopropyl alcohol can be added to the FAE layer.

The composition is then mixed in a collection vessel for about 1 hour. After the vessel settles for about 24 hours, a bottom layer that forms is removed and discarded, leaving behind a FAE layer. Any foam that had been collected during fermentation is mixed with the remaining FAE layer.

After this final layer separation step, the growth by-products, either in purified or unpurified form, can be analyzed to confirm, for example, the presence of FAEs. The FAE layer produced by Meyerozyma guilliermondii is analyzed using LC-MS to demonstrate a molecular weight match with the known molecular weight of FAE molecules. Thin layer chromatography analysis can then be used to show to presence of fatty acid chain(s), confirming the presence of FAEs. Gas chromatography analysis is used to show matching retention times to known FAEs, demonstrating the same chemical properties such as polarity, boiling point, and solubility to known FAEs.

1. A method for producing a fatty acid ester, the method comprising:
a) obtaining an inoculum of a Meyerozyma sp. yeast;
b) inoculating a fermentation reactor having contained therein a liquid growth medium with the inoculum to produce a yeast culture; and
c) growing the yeast culture under appropriate dO₂ concentration, temperature, and pH for a period of time necessary for fermentation and production of the fatty acid ester.

2. The method of claim 1, wherein the fatty acid ester is selected from a fatty acid methyl ester, fatty acid ethyl ester, fatty acid propyl ester, fatty acid butyl ester, fatty acid isopropyl ester and fatty acid hexyl ester.

3. The method of claim 1, wherein the Meyerozyma sp. is Meyerozyma guilliermondii.

4. The method of claim 1, wherein step a) comprises growing a yeast seed culture to a concentration of 1×10⁵ to 1×10¹³ CFU/mL in a liquid culture medium, under continuous aeration and agitation at a temperature of about 20° C. to about 30° C. for about 30 to about 54 hours.

5-9. (canceled)

10. The method of claim 1, wherein a pH adjuster is added to the liquid growth medium to achieve a pH of about 5.0 to about 7.5.

11-13. (canceled)

14. The method of claim 1, wherein step c) occurs at about 20° C. to about 30° C.

15. The method of claim 1, wherein step c) occurs at a d02 level of about 35% to about 65%.

16. The method of claim 1, wherein step c) occurs for about 72 hours to about 120 hours.

17. The method of claim 1, wherein step c) further comprising collecting fatty acid esters as they are produced throughout fermentation.

18. The method of claim 1, further comprising, after step c), allowing the yeast culture to set undisturbed for 20 to 30 hours, wherein a layer comprising a fatty acid ester forms, and wherein the fatty acid ester layer is collected from the yeast culture.

19. (canceled)

20. The method of claim 18, further comprising adding alcohol to the fatty acid ester layer and mixing the composition for about 15 minutes to about 30 hours to enhance esterification, wherein a second fatty acid ester layer forms.

21. The method of claim 20, wherein the alcohol used for esterification is methanol, ethanol, butanol, hexanol, propanol or isopropyl alcohol.

22. The method of claim 20, further comprising extracting the second fatty acid ester layer.

23-27. (canceled)

28. A yeast culture comprising yeast, a liquid growth medium, and at least one fatty acid ester, said fatty acid ester being selected from fatty acid methyl ester, fatty acid ethyl ester, fatty acid propyl ester, fatty acid butyl ester, fatty acid isopropyl ester and fatty acid hexyl ester; wherein the yeast is a Meyerozyma sp.

29. (canceled)

30. The yeast culture of claim 28, wherein the Meyerozyma sp. is Meyerozyma guilliermondii.

31. (canceled)

32. The yeast culture of claim 28, wherein the concentration of fatty acid ester is 0.5 g/L to 100 g/L.

33. (canceled)
